(19) United States
(12) Patent Application Publication
(10) Pub. No.: US 2013/0326803 A1
(43) Pub. Date: Dec. 12, 2013

(54) Personal Cleansing Apparatus

(71) Applicant: Kadi Maleki, Jacksonville Beach, FL (US)

(72) Inventor: Kadi Maleki, Jacksonville Beach, FL (US)

(21) Appl. No.: 13/489,535

(22) Filed: Jun. 6, 2012

(51) Int. Cl. A61H 35/00

(52) U.S. Cl. 4/448

(57) ABSTRACT

A personal cleansing apparatus includes a sprayer housing having inlet and outlet ends, the inlet end being operatively connected to the water inlet source. A first valve assembly is situated adjacent the inlet end of the sprayer housing, the first valve assembly operable between an open configuration that allows water to pass downstream and a closed configuration preventing water from passing downstream. An actuator is operatively coupled to the first valve assembly and configured to selectively move the first stopper between closed and open configurations. A second valve assembly is situated adjacent the sprayer housing outlet end, the second valve assembly configured to move between an open configuration when a predetermined amount of water pressure is experienced and a closed configuration when less than a predetermined amount of water pressure is experienced. Water is permitted to pass downstream when the second valve assembly is at the open configuration.

### BACKGROUND OF THE INVENTION

This invention relates generally to sprayer cleaning devices and, more particularly, to a personal hygiene cleansing apparatus that connects to a toilet water source and is configured to give a user one handed use in cleaning personal body areas while sitting on a toilet seat.

Some individuals desire to clean themselves after using the restroom more thoroughly than just with toilet paper. For instance, the person may desire to clean their bottom areas with water such as in the shower. One specific example of the need for a higher level of cleansing is persons of Muslim faith. Per Islamic beliefs, all followers must cleanse themselves before prayer. Muslims are to pray five times each day. However, since it is not feasible to shower before each time of prayer, followers of Islamic beliefs need an alternative means of cleansing themselves after each time they use the toilet.

Currently, many Islamic households have a separate faucet adjacent the toilet and a long hose extending from the faucet with which a person may clean themselves after using the toilet. Although assumably effective for its intended purpose, the existing hose and faucet configuration has a major disadvantage of cross-contamination. Namely, a user holds the hose in one hand and actuates the faucet with the other hand. The user continues to hold the hose in one hand while washing herself with the other. Finally, the user must then turn off the faucet with the hand that was used for cleaning—causing the faucet to be cross-contaminated.

Therefore, it would be desirable to have a personal cleansing device that allows one handed activation and deactivation of a water stream for personal cleansing without ever having to turn off a faucet with a contaminated hand. Further, it would be desirable to have a personal cleansing device in which water from a toilet water supply is actuated to flow from a spray housing upon actuation of a push button actuator and deactivated by releasing the button.

### SUMMARY OF THE INVENTION

A personal cleansing apparatus for connection to a water inlet source according to the present invention includes a sprayer housing having opposed inlet and outlet ends and defining an interior area, the sprayer housing inlet end being operatively connected to the water inlet source such that water is received into the sprayer housing interior area. A first valve assembly is situated generally adjacent the inlet end of the sprayer housing, the first valve assembly being selectively operable between an open configuration that allows water to pass downstream through the interior area and a closed configuration preventing water from passing downstream. An actuator is operatively coupled to the first valve assembly and configured to selectively move the first stopper between the closed and open configurations. A second valve assembly is situated generally adjacent the outlet end of the sprayer housing, the second valve assembly configured to move between an open configuration when a predetermined amount of water pressure is experienced and a normally closed configuration when less than a predetermined amount of water pressure is experienced. Accordingly, water is permitted to pass downstream of the second valve assembly and through the sprayer housing outlet when the second valve assembly is at the open configuration.

Therefore, a general object of this invention is to provide a personal cleansing apparatus for connection to a toilet water supply that enables a user to cleanse personal areas of the body after using the toilet.

Another object of this invention is to provide a personal cleansing apparatus, as aforesaid, having a sprayer housing that selectively mounts to a wall adjacent a toilet for single handed use that avoids cross contamination of plumbing fixtures.

Still another object of this invention is to provide a personal cleansing apparatus, as aforesaid, that does not require a water sources to be turned on or off, but rather that may be actuated with a push button.

Yet another object of this invention is to provide a personal cleansing apparatus, as aforesaid, that is particularly useful by persons of Muslim faith who must cleanse themselves prior to praying multiple times per day.

A further object of this invention is to provide a personal cleansing apparatus, as aforesaid, having a curvature that enhances a user's ability to cleanse personal areas of the body while seated on a toilet seat.

A still further object of this invention is to provide a personal cleansing apparatus, as aforesaid, that is easy to use and cost-effective to manufacture.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a personal cleansing apparatus installed adjacent a toilet according to a preferred embodiment of the present invention;

FIG. 2 is a side view of the spray housing of the personal cleansing apparatus as in FIG. 1;

FIG. 3a is a front view of the spray housing of the personal cleansing apparatus as in FIG. 1;

FIG. 3b is a sectional view taken along line 3b-3b of FIG. 3a;

FIG. 4 is an isolated view on an enlarged scale taken from FIG. 3b;

FIG. 5 is an isolated view on an enlarged scale taken from FIG. 3b;

FIG. 6 is a perspective view of a mounting bracket according to the present invention; and

FIG. 7 is a perspective view of a personal cleansing apparatus according to another embodiment of the present invention.

### DESCRIPTION OF THE PREFERRED EMBODIMENT

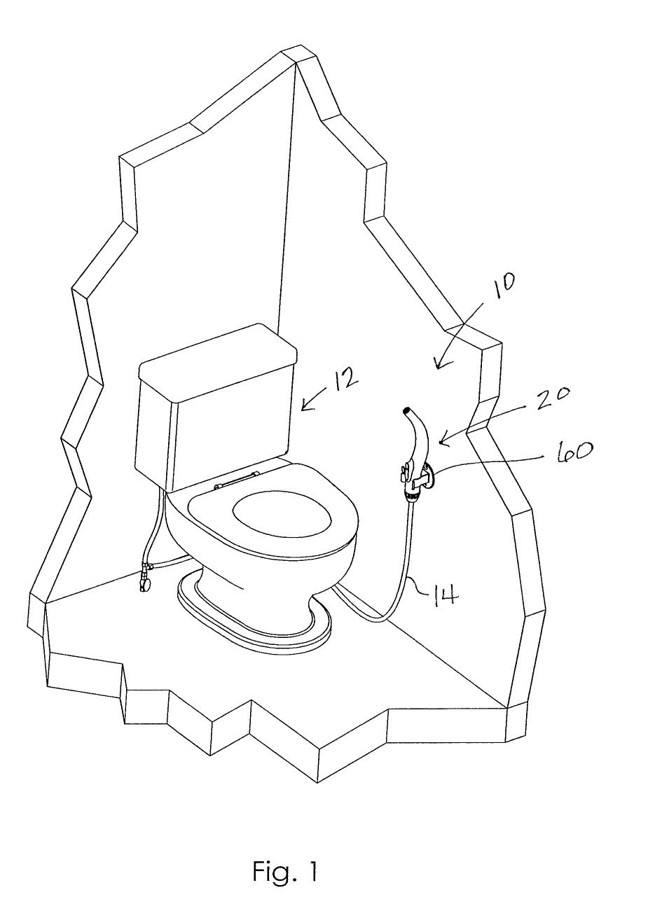

A personal cleansing apparatus according to a preferred embodiment of the present invention will now be described with reference to FIGS. 1 to 5 of the accompanying drawings. The personal cleansing apparatus 10 includes a spray housing 20, a connector hose 14, and a wall mounting bracket 60.

The spray housing 20 includes a continuous side wall extending between opposed inlet 22 and outlet 24 ends. The spray housing, therefore, may include a generally cylindrical configuration having open ends although other configurations may be possible. The spray housing 20 may have the appearance of a wand. An ergonomically configured handgrip 26 may be integrally formed by the spray housing 20 adjacent the inlet end 22. The spray housing 20 continuous side wall defines an interior area. The spray housing 20 may include a generally annular configuration as shown in FIG. 2. Specifically, the annular portion of the spray housing 20 may extend from the handgrip 26 to the outlet end 24. Further, the annular portion of the spray housing 20 may include a progressively decreasing inner diameter between the first valve assembly and the second valve assembly. The decreasing diameter of the spray housing 20 may increase the pressure of the water, which is an advantage for using the present invention for cleansing one's body.

As shown in FIG. 1, the inlet end 22 of the spray housing 20 may be operatively connected to a toilet water inlet source with a hose 14 and configured for fluid flow therebetween. Water is automatically in fluid communication with the inlet end 22 and interior area of the spray housing 20 as will be further described later. In one embodiment, the inlet end 22 of the spray housing 20 may be coupled to a water source independent of the toilet, such as to a plumbing fixture 70 having hot and cold water settings mounted to a wall adjacent the toilet (FIG. 7).

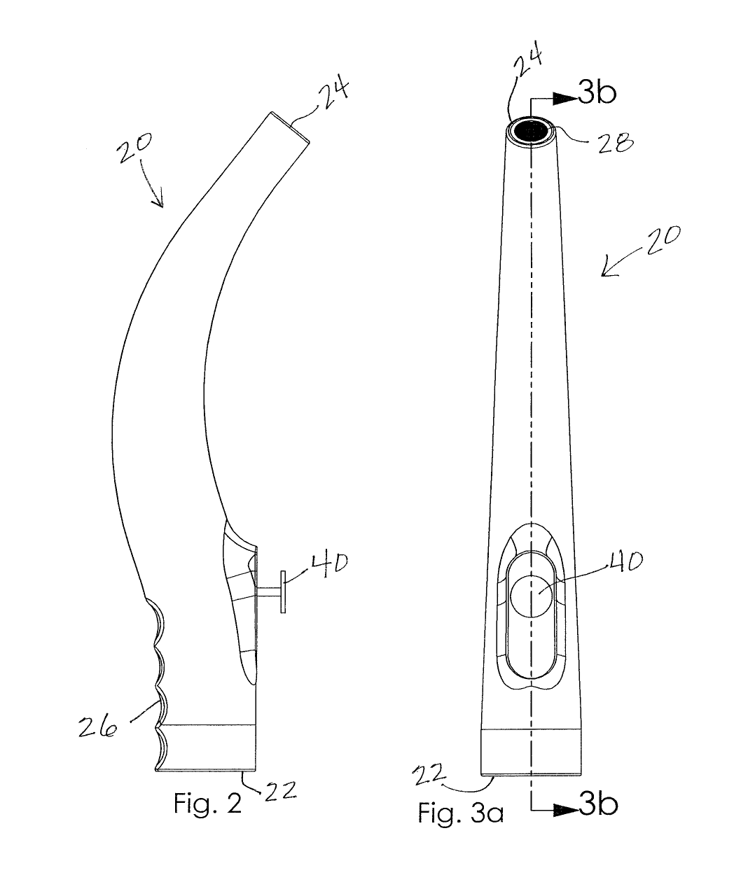

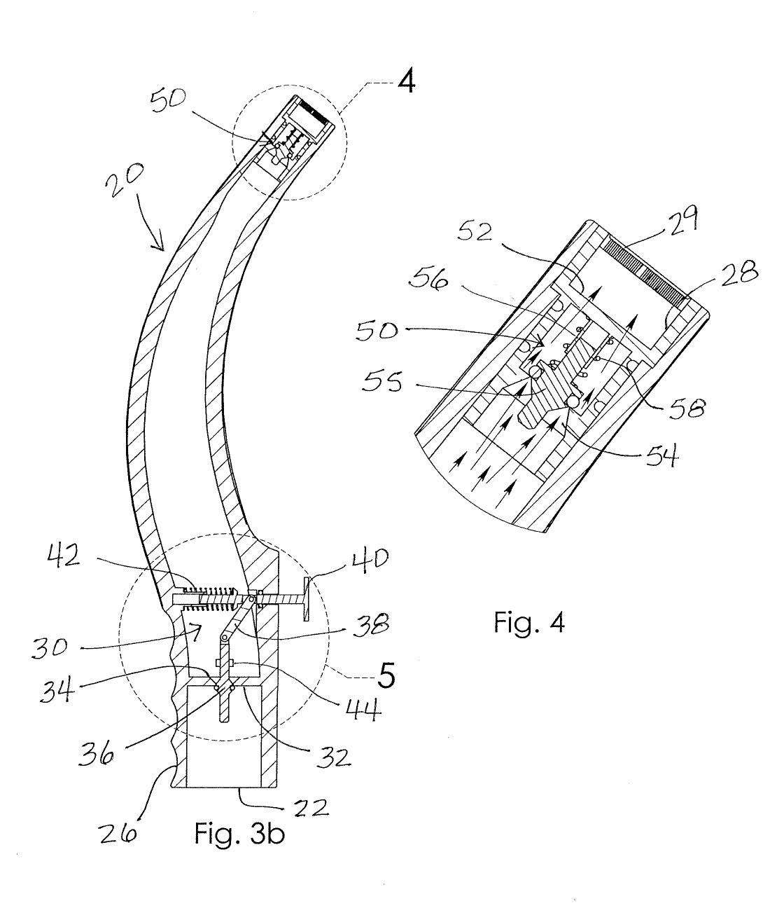

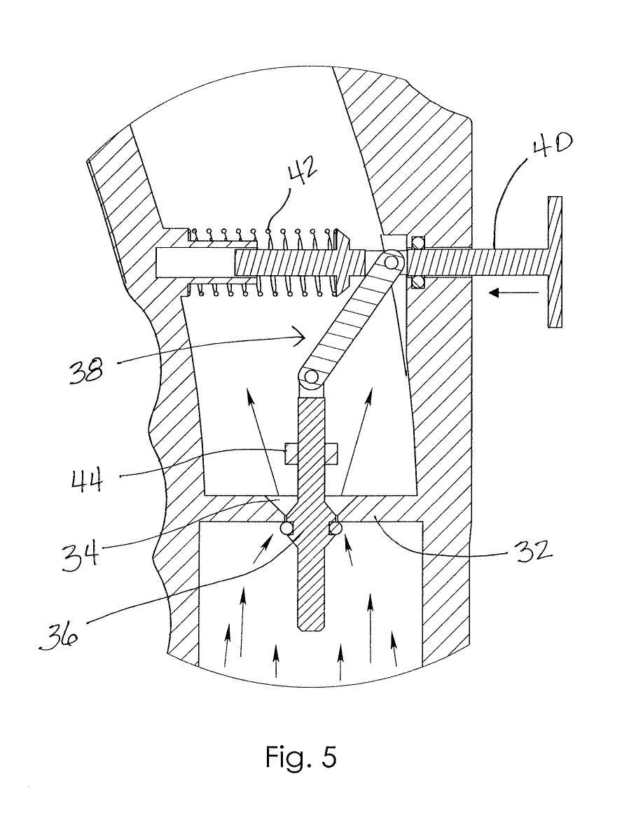

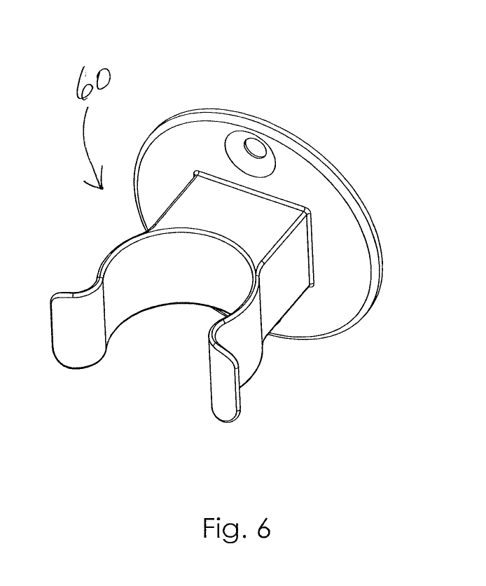

A first valve assembly 30 is situated in the interior area of the spray housing 20 generally adjacent the inlet end 22 (FIG. 3b). The first valve assembly 30 is selectively operable between a normally closed configuration that does not allow water received into the housing interior area from the toilet water inlet source to pass downstream (FIGS. 3b and 5) and an open configuration that allows water to pass downstream.

More particularly, the first valve assembly 30 includes a first barrier 32 that extends between inner surface(s) of the spray housing 20 (FIG. 3b). The first barrier 32 defines a first aperture 34 through which water may flow at the first valve open configuration. As shown, the first barrier 32 may have an integral construction with the housing 20 or may be a separate component. The first valve assembly 30 may also include a first stopper 36 having a configuration that is complementary to the first barrier aperture 34. Further, the first valve assembly 30 may include a linkage 38 that is operatively coupled to the actuator and to the first stopper 36. The linkage 38 is configured to move the first stopper 36 between the closed configuration at which the first stopper 36 is nested in the first aperture 34 and the first valve open configuration at which the first stopper 36 is displaced from the first aperture 34. In operation, water entering the spray housing 20 through the inlet end 22 is prevented from passing through the first barrier aperture 34 and then downstream in the spray housing 20 so long as the first stopper 36 is nested in the first barrier aperture 34. This represents the situation when the spray housing is not in use.

An actuator 40, such as a button, is operatively coupled to the first valve assembly 30 and is configured to selectively move the first stopper 36 between the closed and open configurations when engaged by a user. The actuator 40 is coupled to a linkage 38 that is configured to connect the actuator 40 to the first stopper 36. Further, the actuator 40 includes a spring 42 that is configured to bias the actuator in a normally unactuated (i.e. released) configuration at which the first valve assembly 30 is at the closed configuration. In addition, a guide member 44 is positioned adjacent the first barrier 32 and configured to slidably receive a portion of the first stopper 36, the guide member 44 being positioned to keep the first stopper 36 properly aligned with the first barrier aperture 34. The first valve assembly 30 may also include one or more O-rings strategically positioned to seal the first stopper 36 relative to the first barrier aperture 34 at the closed configuration (FIG. 3b).

A second valve assembly 50 is situated in the interior area of the spray housing 20 generally adjacent the outlet end 24 and displaced from the first valve assembly 30 (FIGS. 3b and 4). The second valve assembly 50 is configured to move between an open configuration as a result of experiencing a predetermined amount of water pressure and a normally closed configuration when less than a predetermined amount of water pressure is experienced. The second valve assembly 50 includes a second barrier 52 configured to extend between inner surfaces of the sprayer housing continuous side wall. The second barrier 52 presents a blockage of fluid flow through the interior area of the spray housing 20 between inlet and outlet ends. The second barrier 52 defines an aperture 54 (also referred to as a second aperture or second barrier aperture) through which water may pass when the second valve assembly 50 is at the open configuration as will be further described below.

The second valve assembly further includes a stopper, also referred to as a second stopper 55 or second valve stopper. The second stopper 55 includes a configuration complementary to that of the second barrier aperture 52. The second stopper 55 is nested and sealed in the second barrier aperture 52 at the second valve closed configuration (FIG. 4). Strategically positioned O-rings may also be used to augment this seal.

The second valve assembly 50 includes a guide channel 56 mounted in the sprayer housing adjacent the second barrier 52. The guide channel 56 is configured and situated to slidably receive a portion of the second stopper 55 so as to maintain the second stopper 55 in proper alignment with the second barrier aperture 52. A second valve spring 58 is operatively coupled to the second stopper 55 and to the guide channel 56 and configured to bias the second stopper 55 toward the second valve closed configuration (FIG. 4). The second stopper 55 is configured to be moved automatically to the open configuration and into slidable engagement with the guide channel 56 when a predetermined amount of water pressure bears against the second stopper 55. For instance, when the first valve assembly 30 is opened and water is allowed to flow through the interior area of spray housing 20, the pressure of that fluid flow automatically urges the second valve assembly 50 to the second valve open configuration, allowing the water to flow through the second valve and out the outlet end 24 of the spray housing 20.

The inlet end 22 of the spray housing 20 may include a nozzle 28 configured to direct the flow of water in a focused or patterned stream. The nozzle 28 is situated downstream from the second valve assembly 50 such that water may be expelled through the nozzle 28 only when it has first passed through the second valve assembly 50 as described above. The nozzle 28 may include an aerator 29 configured to inject a water stream with water. The aerator 29 has the effect of saving water and increasing the perceived pressure of the water stream.

In another aspect of the invention, the spray housing 20 includes a handgrip 26 generally adjacent the inlet end 22 and nozzle 28. The handgrip 26 may be integrated into the sprayer housing side wall or may be mounted thereto. Specifically, the handgrip 26 may include a configuration that is complementary to a person's fingers and may extend from a position immediately adjacent the open inlet end 22 of the spray housing 20 to a point immediately opposed from the nozzle 28 (FIG. 3b). The handgrip 26 is positioned such that a user may hold the spray housing 20 and operate the actuator 40 in a single handed operation.

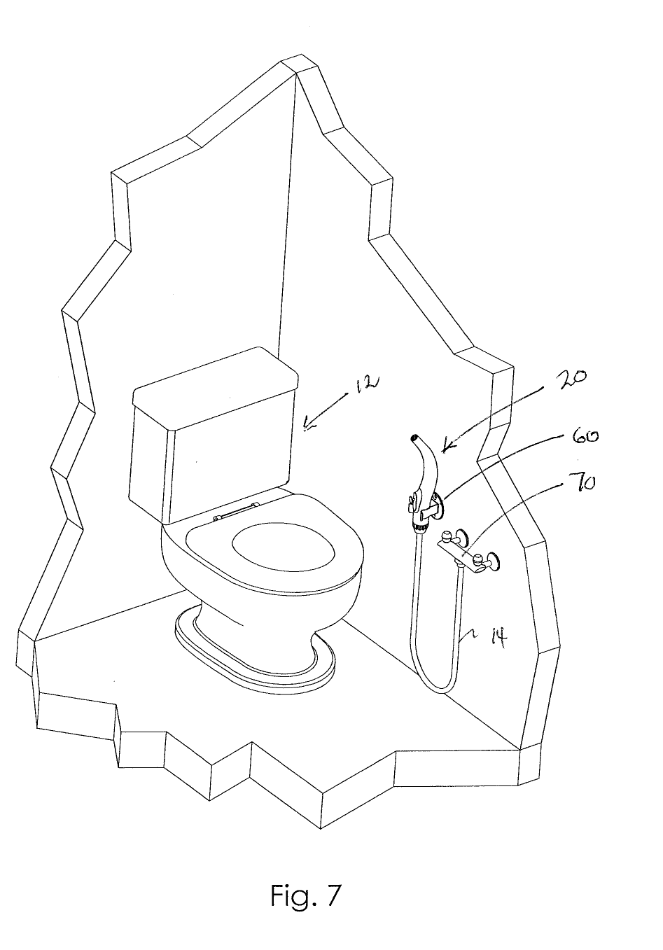

In yet another aspect of the invention, the personal cleansing apparatus 10 includes a bracket 60 mountable to a wall and configured to secure the spray housing 20 when not in use (FIGS. 1 and 6). The mounting bracket 60 may include a pair of inwardly biased spring steel clips arranged in a generally U-shaped configuration into which the spray housing 20 may be inserted in a friction fit engagement.

In use, the personal cleansing apparatus 10 may be installed in a bathroom adjacent an existing toilet 12. The mounting bracket 60 may be coupled to a wall using traditional mounting hardware, such as screws or the like. When not in use, the spray housing 20 may be removably coupled to the mounting bracket 60. A hose 14 may be in fluid communication with the inlet end 22 of the sprayer housing. An opposite end of the hose 14 may be coupled for fluid communication with the water source for the toilet 12 using traditional plumbing hardware as shown in FIG. 1.

When use of the spray housing 20 is desired, such as to clean personal areas of one's body after using the toilet 12, the spray housing 20 may be removed from the mounting bracket 60 and held by the handgrip 26 to position the outlet end 24 as desired. When ready to release a water stream, the user may press the actuator 40, such as with the thumb of the same hand with which the handgrip 26 is being held. Engaging the actuator 40 causes the first valve assembly 30 to move to the open configuration which allows water from the hose 14 to flow downstream through the spray housing interior area. Consequently, the pressure of the water flow urges the second valve assembly 50 to its open configuration which enables the water to flow outwardly through the nozzle 28 and outlet end 24. When finished cleansing, the actuator 40 may be released and the first valve assembly spring 42 causes the first valve assembly 30 to close. Finally, the spray housing 20 may be returned to the mounting bracket 60.

It is understood that while certain forms of this invention have been illustrated and described, it is not limited thereto except insofar as such limitations are included in the following claims and allowable functional equivalents thereof.

1. A personal cleansing apparatus for connection to a water inlet source, comprising:
a sprayer housing having opposed inlet and outlet ends and defining an interior area, said sprayer housing inlet end being operatively connected to the water inlet source such that water is received into said sprayer housing interior area;
a first valve assembly situated generally adjacent said inlet end of said sprayer housing, said first valve assembly selectively operable between a first valve open configuration that allows water to pass downstream through said interior area and a first valve closed configuration preventing water from passing downstream;
an actuator operatively coupled to said first valve assembly and configured to selectively move said first stopper between said closed and open configurations;
a second valve assembly situated generally adjacent said outlet end of said sprayer housing, said second valve assembly configured to move between a second valve open configuration when urged by a predetermined amount of water pressure and a normally second valve closed configuration when less than a predetermined amount of water pressure is experienced; and
whereby water is permitted to pass downstream of said second valve assembly and through said sprayer housing outlet when said second valve assembly is at said second valve open configuration.

2. The apparatus as in claim 1, wherein said second valve assembly is automatically at said second valve open configuration when said first valve assembly is at said first valve open configuration.

3. The apparatus as in claim 1, wherein said first valve assembly includes:
a first barrier configured to extend between inner surfaces of said sprayer housing, said first barrier defining a first barrier aperture;
a first stopper having a configuration complementary to said first barrier aperture; and
a linkage operatively connecting said actuator to said first stopper and configured to move said first stopper between said closed configuration at which said first stopper is nested in said first barrier aperture and said first valve open configuration at which said first stopper is displaced from said first barrier aperture.

4. The apparatus as in claim 3, wherein said first valve assembly includes:
a guide member situated adjacent said first barrier, said first stopper in slidable communication with said guide member so as to maintain said first stopper in alignment with said first barrier aperture; and
an O-ring seal coupled to said first stopper and configured to seal said first barrier aperture at said first valve closed configuration.

5. The apparatus as in claim 3, wherein said actuator includes a spring configured to bias said actuator in a normally unactuated configuration at which said first valve assembly is at said first valve closed configuration.

6. The apparatus as in claim 3, wherein said second valve assembly includes:
a second barrier configured to extend between inner surfaces of said sprayer housing, said second barrier defining a second barrier aperture; and
a second stopper having a configuration complementary to said second barrier aperture, said second stopper being nested in said second barrier aperture at said second valve closed configuration and being displaced from said second barrier aperture at said second valve open configuration.

7. The apparatus as in claim 6, wherein said second valve assembly includes:
a guide channel mounted in said sprayer housing adjacent said second barrier, said second stopper being in slidable communication with said guide channel so as to maintain said second stopper in alignment with said second barrier aperture; and
a spring operatively coupling said second stopper to said guide channel and configured to bias said second stopper toward said second valve closed configuration.

8. The apparatus as in claim 6, wherein said second stopper is configured to move to said second valve open configuration when urged by a predetermined amount of water pressure upstream from said second stopper.

9. The apparatus as in claim 1, further comprising a nozzle coupled to said sprayer housing outlet end, said nozzle situated downstream from said first and second valve assemblies.

10. The apparatus as in claim 10, wherein said nozzle includes an aerator.

11. The apparatus as in claim 6, wherein said sprayer housing includes a handgrip generally adjacent said inlet end and said actuator such that said actuator is selectively actuated by a user with the same hand with which said handgrip is held.

12. The apparatus as in claim 1, wherein said sprayer housing includes a handgrip generally adjacent said inlet end and said actuator such that said actuator is selectively actuated by a user with the same hand with which said handgrip is held.

13. The apparatus as in claim 1, further comprising a mounting bracket configured to selectively retain said spray housing.

14. The apparatus as in claim 1, wherein said spray housing inlet end is operatively coupled to said water inlet source with a hose.

15. The apparatus as in claim 1, wherein said spray housing include a portion having a generally annular configuration.

16. The apparatus as in claim 15, wherein said spray housing includes a progressively decreasing inner diameter between said first valve assembly and said second valve assembly.
